(19) United States
(12) Patent Application Publication
(10) Pub. No.: US 2026/0115378 A1
(43) Pub. Date: Apr. 30, 2026

(54) SYSTEM AND METHODS FOR CONTROLLED ADMINISTRATION OF UNSUSPENDED PARTICLES

(71) Applicant: UNandUP, LLC, Saint Louis, MO (US)

(72) Inventors: Francis M. Creighton, St. Louis, MO (US); Michael E. Sabo, Prairie du Rocher, IL (US); Leah J. Morrell, St. Louis, MO (US)

(21) Appl. No.: 18/930,822

(22) Filed: Oct. 29, 2024

(51) Int. Cl. A61M 5/142; A61M 5/145

(52) U.S. Cl. CPC A61M 5/14216; A61M 5/14526

(57) ABSTRACT

Disclosed herein are systems and methods for controlled administration of unsuspended particles. The system may include an agitation device, at least one reservoir, an infusion mechanism, and a delivery conduit. The system may be configured to infuse unsuspended particles to a patient that are heterogeneously or homogenously dispersed.

### CROSS-REFERENCE TO RELATED APPLICATIONS

This application claims the benefit of U.S. Application No. 63/535,442, filed Aug. 30, 2023, the entire contents of which are incorporated herein by reference in their entirety.

### FIELD OF DISCLOSURE

The present disclosure relates to systems and methods for controlled administration of unsuspended particles. The system may include an agitation mechanism, an infusion mechanism, at least one reservoir, and a delivery conduit. The system may be configured to infuse unsuspended particles to a patient that are heterogeneously or homogenously dispersed.

### BACKGROUND

In recent years, nanoparticles have emerged as important players in modern medicine, with applications ranging from contrast agents in medical imaging to carriers for gene delivery into individual cells. Nanoparticles have a number of properties that distinguish them from bulk materials simply by virtue of their size, such as chemical reactivity, energy absorption, and biological mobility. In the medical field, one large subset of nanoparticles are magnetic nanoparticles (MNPs). Magnetic nanoparticles (MNPs), which include magnetic iron oxide nanoparticles, have enormous potential for many biomedical applications, such as biomolecule separation, MRI imaging, hyperthermia, iron replenishment, and tumor embolization. MNPs may be used as carriers of drugs, nucleic acids, peptides and other biologically active compounds. Intravenous injection is the commonly used approach for administration of nanoparticles.

Nanoparticle infusion systems known in the art require specifically created suspensions to maintain homogenously dispersed particles for infusion into a patient or equipment for analyzing the particles. Some nanoparticle applications require magnetic nanoparticles; however, suspensions of the MNPs have required thick buffer layers to maintain homogenous dispersion of magnetic nanoparticles. In many cases, the thicker buffer layers reduce the effectiveness of the magnetic core of the nanoparticles and magnetization of the overall nanoparticle, thereby requiring the use of much stronger magnets to control the nanoparticles as desired.

Therefore, there is a need for a system to homogenously disperse unsuspended nanoparticles and control administration of unsuspended particles. Particularly for magnetic nanoparticles with thinner buffer layers or coatings.

### SUMMARY

Provided herein is a system for controlled administration of unsuspended particles. The system may include at least one reservoir operable to contain a plurality of unsuspended particles and/or a carrier fluid, an agitation mechanism operable to agitate the unsuspended particles in the at least one reservoir, and a delivery conduit in fluid communication with the at least one reservoir. In an aspect, the agitation mechanism provides mechanical agitation to the unsuspended particles to homogenously disperse the plurality of unsuspended particles. In another aspect, the at least one reservoir is a barrel of a syringe. In some aspects, the syringe includes a plunger operable to expel the plurality of unsuspended particles and/or the carrier fluid from the barrel into the delivery conduit. In an aspect, the agitation mechanism is a rocker plate coupled to the barrel. In another aspect, the system includes one or more agitation bodies within the barrel of the syringe. In an aspect, the rocker plate may be operable to agitate the one or more agitation bodies causing mechanical and homogenous dispersion of the unsuspended particles in the barrel.

In another aspect, the agitation mechanism may be a cycling mechanism. The cycling mechanism may include a first syringe having a barrel and a plunger and a second syringe having a barrel and a plunger. The first syringe may be in fluid communication with the second syringe. The cycling mechanism may also include a third syringe having a barrel and a plunger. The barrel of the third syringe may be the at least one reservoir containing the carrier fluid. In an aspect, the first syringe and the second syringe contain the plurality of unsuspended particles. In some aspects, the system may include a manifold in fluid communication with the first syringe, the second syringe, the third syringe and the delivery conduit. The plunger of the first syringe and the plunger of the second syringe may be operable to compress and/or decompress to cycle the unsuspended particles between the first syringe and the second syringe through the manifold causing mechanical and homogenous dispersion of the unsuspended particles. The plunger of the third syringe may be operable to be compressed and expel the carrier fluid into the manifold. In an aspect, the carrier fluid in the manifold may entrain some of the plurality of unsuspended particles, thereby providing the entrained unsuspended particles to the delivery conduit.

In an additional aspect, the agitation mechanism may be a magnetic mixer assembly. The magnetic mixer assembly may include a magnetic mixer fan. The at least one reservoir may be a syringe having a barrel and a plunger. In an aspect, the magnetic mixer assembly may be coupled to an interior of the barrel of the syringe. The system may further include an external magnet subassembly configured to be rotated causing a change in a magnetic field operable to rotate the magnetic mixer fan. The rotation of the magnetic mixer fan may cause mechanical and homogenous dispersion of the unsuspended particles in the barrel of the syringe. The plunger may be operable to expel the homogenously dispersed unsuspended particles and the carrier fluid into the delivery conduit.

In another aspect, the agitation mechanism may be a peristaltic pump. The carrier fluid may be contained in the at least one reservoir and the unsuspended particles may be contained in a peristaltic reservoir. The peristaltic pump may provide a pressure to the unsuspended particles in the peristaltic reservoir causing the unsuspended particles to be homogenously dispersed. In an aspect, the at least one reservoir is a syringe having a barrel and a plunger in fluid communication with the peristaltic reservoir. The plunger may be operable to provide the carrier fluid to the peristaltic reservoir and entrain the homogenously dispersed unsuspended particles. In an aspect, the peristaltic reservoir is in fluid communication with the delivery conduit. The entrained unsuspended particles and the carrier fluid may be delivered to the delivery conduit by the pressure provided by the peristaltic pump.

In an aspect, the unsuspended particles may be magnetic nanoparticles and the carrier fluid may be saline.

Further provided herein is a system for controlled administration of unsuspended particles. The system may include at least one reservoir containing a plurality of unsuspended particles, a carrier fluid, and one or more agitation bodies. The system may further include an agitation mechanism operable to mechanically agitate the one or more agitation bodies causing the plurality of unsuspended particles to be homogenously dispersed. The system may further include a delivery conduit in fluid communication with the at least one reservoir. The delivery conduit may be operable to deliver the homogenously dispersed unsuspended particles and the carrier fluid to a patient. In an aspect, the agitation mechanism may be a rocker plate coupled to the at least one reservoir.

Further provided herein is a system for controlled administration of unsuspended particles. The system may include a first reservoir and a second reservoir containing a plurality of unsuspended particles. The first reservoir may be in fluid communication with the second reservoir. The first reservoir and the second reservoir may also be in fluid communication with a manifold. The system may include a third reservoir containing a carrier fluid. The third reservoir may be in fluid communication with the manifold. The system may further include a delivery conduit in fluid communication with the manifold. The unsuspended particles may be cycled between the first reservoir and the second reservoir through the manifold via a pressure source causing homogenous dispersion of the plurality of unsuspended particles. The carrier fluid may be expelled from the third reservoir via a second pressure source and entrain some of the plurality of unsuspended particles passing through the manifold. The entrained unsuspended particles in the carrier fluid may be delivered to a patient via the delivery conduit.

Further provided herein is a system for controlled administration of unsuspended particles. The system may include at least one reservoir containing a plurality of unsuspended particles and a carrier fluid. The system may include a magnetic mixer fan assembly having a magnetic mixer fan and coupled within the at least one reservoir. An external magnet assembly may be located near an exterior of the at least one reservoir. The system may include a delivery conduit in fluid communication with the at least one reservoir. The rotation of the external magnet assembly may cause rotation of the magnetic mixer fan. The rotation of the magnetic mixer fan may cause fluidic mixing of the plurality of unsuspended particles in the at least one reservoir. The fluidically mixed unsuspended particles may be homogenously dispersed in the carrier fluid. The homogenously dispersed unsuspended particles and the carrier fluid may be expelled from the at least one reservoir to the deliver conduit via a pressure source.

Further provided herein is a system for controlled administration of unsuspended particles. The system may include a first reservoir containing a carrier fluid. The first reservoir may be in fluid communication with a peristaltic reservoir containing a plurality of unsuspended particles. The system may include a peristaltic pump in fluid communication with the peristaltic reservoir. The peristaltic pump may be operable to provide a pressure to the peristaltic reservoir, thereby homogenously dispersing the plurality of unsuspended particles. The carrier fluid may be provided to the peristaltic reservoir via a pressure source. The carrier fluid may entrain the unsuspended particles in the peristaltic reservoir. The carrier fluid and the entrained unsuspended particles may be provided to the delivery conduit via the pressure provided by the peristaltic pump.

Further provided herein is a method for controlled administration of unsuspended particles. The method may include providing a plurality of unsuspended particles to at least one reservoir, providing agitation to the plurality of unsuspended particles in the at least one reservoir via an agitation mechanism, entraining the plurality of unsuspended particles in a carrier fluid, and delivering the plurality of unsuspended particles entrained in the carrier fluid to a patient or infusion apparatus via a delivery conduit.

Other aspects and iterations of the invention are described more thoroughly below.

### BRIEF DESCRIPTION OF FIGURES

The patent or application file contains at least one drawing executed in color. Copies of this patent or patent application publication with color drawing(s) will be provided by the Office upon request and payment of the necessary fee.

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1A is a system for controlled administration of unsuspended particles in one example.

FIG. 1B is a system for controlled administration of unsuspended particles in one example

FIG. 1C is a system for controlled administration of unsuspended particles in one example.

FIG. 1D is a system for controlled administration of unsuspended particles in one example.

FIG. 2A is a system for controlled administration of unsuspended particles in one example.

FIG. 2B is a system for controlled administration of unsuspended particles in one example.

FIG. 2C is a system for controlled administration of unsuspended particles in one example.

FIG. 2D is a system for controlled administration of unsuspended particles in one example.

FIG. 3A is a system for controlled administration of unsuspended particles in one example.

FIG. 3B is a system for controlled administration of unsuspended particles in one example

FIG. 3C is a magnetic mixer assembly in one example.

FIG. 3D is an exploded view of a magnetic mixer assembly in one example.

FIG. 4 is a system for controlled administration of unsuspended particles in one example.

FIG. 5 is a graph of unsuspended particle delivery in one example.

FIG. 6 is a graph of unsuspended particle delivery in one example.

FIG. 7 is a flowchart of a method for controlling administration of unsuspended particles in one example.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The terms “nanoparticle”, “particle,” “bead,” and “particulate” may be used interchangeably in this disclosure. Nanoparticles are materials with overall dimensions in the nanoscale, (e.g., under 100 nm). Some definitions expand this to 500 nm (or even 1000 nm), however the definition here of nanoparticle will be those particles with overall dimensions under 100 nm. For this application, fine particles will have a dimension of 100-2,500 nm and course particles will have a dimension of 2,500 to 10,000 nm. Particles may also include embolization beads having a dimension of 50,000 nm to 250,000 nm. Embolization beads include magnetic embolization beads. Collectively nanoparticles, fine particles, course particles, and embolization beads will be referenced within this application as particles (although other known definitions of these terms use sizes of micro-particle that distinguish between micro-particles and nanoparticles).

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Intravenous injection is the commonly used approach for administration of nanoparticles. However, the systems and methods described herein may be used with intraarterial and intrathecal administration, as well as intravenous injection. In these applications the nanoparticle typically must be homogeneously distributed within a carrier medium (e.g., saline) for controlled administration of the designated nanoparticle therapy. This is generally accomplished by formulating the nanoparticle as a suspended solid (or material) within the carrier medium. Suspended solids refer to small solid particles which remain in suspension in a medium (e.g., saline) as a colloid or technically due to motion of the medium. This suspension requirement (often referenced as stability of the particle) limits the design of the nanoparticle. With magnetic nanoparticles (MNPs) this requirement and inclusion of coatings can alter the controllability of the particles, namely increase the magnetic fields and/or forces required to control the particle. For example, with suspended particles, to promote homogenous dispersion the coating layer must increase the distance between cores to mitigate the attractive force of particles above the superparamagnetic size range, thereby reducing the magnetic properties of the particles. This minimum distance varies as a function of core size. The other role of the coating is in reducing the overall density of the entire particle, for both magnetic and non-magnetic particles with high densities relative to the carrier fluid.

Provided herein is a system for controlled administration of unsuspended particles, thereby removing the need to suspend particles for intravenous injection or other applications. The administration of unsuspended particles may be used in the field of medicine or other technology fields such as engineering and agriculture. The system agitates the unsuspended particles to homogenously distribute the unsuspended particles for infusion.

The system described herein includes at least one reservoir containing unsuspended particles, a delivery conduit, an agitation mechanism, and an infusion mechanism (e.g., syringe, etc.). In some examples, at least one reservoir may be a component of the infusion mechanism (e.g., a barrel of a syringe). In some examples, the system may include a carrier fluid. In one example, the carrier fluid and the unsuspended particles may be placed in the same reservoir. In other examples, the unsuspended particles and the carrier fluid may be placed in different reservoirs and the carrier fluid may later be combined (e.g., mixed) with the unsuspended particles.

The agitation mechanism homogenously disperses the unsuspended particles which are then entrained in the carrier fluid. The entrained homogenously dispersed unsuspended particles (e.g., homogenous mixture) may then be delivered to a patient or infusion apparatus via a delivery conduit. In some examples, the unsuspended particles may be heterogeneously dispersed at a desired dispersion (i.e., distribution) profile. The system may be operable to achieve a desired particulate infusion over time, whether the infusion be with an entirely homogenous dispersion of unsuspended particles or a heterogenous dispersion of unsuspended particles.

In some examples, a pressure source may be operable to expel the unsuspended particles and/or carrier fluid from the reservoirs described herein. While specific examples of syringe systems are shown and described herein, the pressure source may be a different pressure source than the plunger of the syringe. In some examples, the pressure source may be any type of pump mechanically or fluidically connected to the system and operable to provide a pressure to move the unsuspended particles and/or carrier fluid through the various components of the system.

In an aspect, the unsuspended particles may be nanoparticles, microparticles, chemicals, metals, or any material with a density different than the density of the carrier fluid. In an example, the unsuspended particles may be magnetic nanoparticles (MNPs). In other examples, the unsuspended particles may be liposomes, proteins, microbubbles, and/or polymer constructs. In some examples, the polymer constructs may be embolization beads such as a polymer linked to a gelatin or other resorbable polymer (e.g., poly acrylic, poly vinyl alcohol, etc.). It will be appreciated that the unsuspended particles may be any particle useful to be infused in a body of a patient and/or infused in another application where homogenous or controlled heterogenous dispersion of particles is desired.

The size of MNPs for injection applications are typically within the range from 1 to 100 nm. In addition, the nanostructures for injection applications are conventionally coated with polymers, such as polyethylene glycol, dextran or silanes, to provide stability and avoid aggregation. Dextran-coated iron oxide Fe₃O₄ nanoparticles sized 80-150 nm as well as dextran-coated MNPs sized 20-40 nm are examples of MNPs used for mononuclear phagocyte system imaging, lymph node and perfusion imaging as well as cellular labelling. Other MNPs having a size range of about 50 nm to about 250 nm may be used with the systems described herein. MNPs may be administered using the systems described herein. Other particle sizes may be used with the systems and methods described herein. For example, liposome sized particles of about 20 μm to about may be used. Furthermore, embolization beads having a size of about 50 μm to about 250 μm may be administered using the systems described herein. Other particle sizes ranging from about 1 nm to about 250 μm may be administered using the systems described herein.

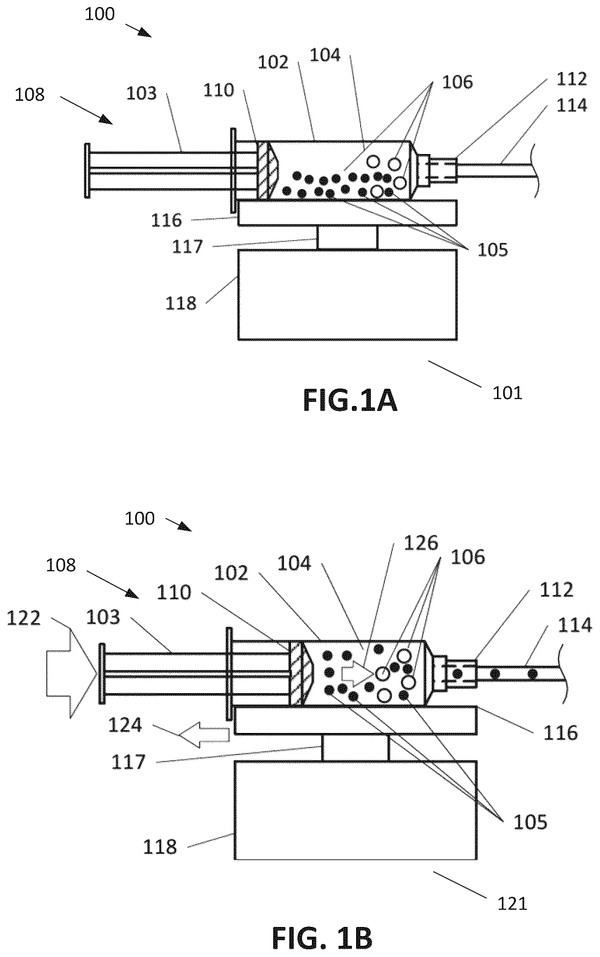

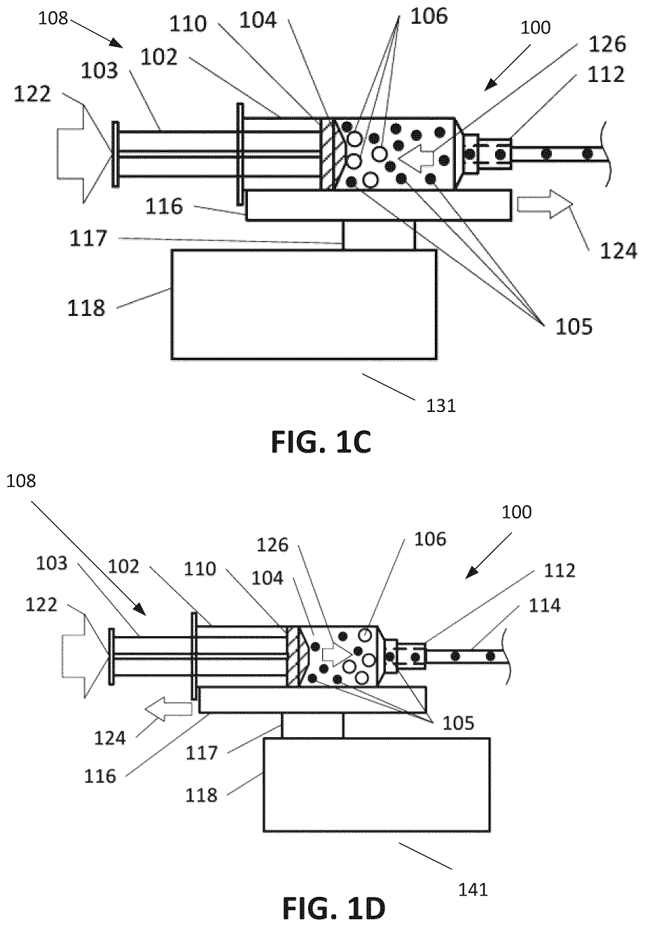

FIGS. 1A-1D illustrate a mechanical agitation system 100 for controlled administration of unsuspended particles. The mechanical agitation system 100 may include an infusion mechanism. In some examples, the infusion mechanism may be a syringe 108. The syringe 108 may have a plunger 103 and a barrel 102 (e.g., reservoir). The barrel 102 may be a cylindrical body. The plunger may have a plunger end 110 that is placed within the barrel 102 (e.g., cylindrical body) of the syringe 108. The plunger 103 may be linearly movable within the barrel 102. In some examples, the plunger may operate as a pressure mechanism for expelling the contents of the syringe into a tube or conduit. The syringe may be in fluid communication with a delivery conduit 114. The delivery conduit 114 may be operable to provide the unsuspended particles to a patient or infusion apparatus. In other examples, the infusion mechanism may comprise any reservoir capable of containing a carrier fluid 104 and unsuspended particles 105 and any mechanism for moving the carrier fluid 104 and unsuspended particles to the delivery conduit 114.

In one example, the syringe 108 may be oriented horizontally, as illustrated, for example, in FIGS. 1A-1D. In another example, the syringe 108 may be oriented vertically. In another example, the syringe 108 may be oriented in any other configuration.

In an aspect, the carrier fluid 104 may be saline. In other examples, other carrier fluids operable to be infused into a patient may be used. In other non-medical applications, other known carrier fluids may be used.

In some aspects, the syringe barrel 102 may be filed with the carrier fluid 104, the unsuspended particles 105, and one or more agitation bodies 106. In some examples, the agitation bodies 106 may be biologically safe, sterile, and/or nonreactive. The agitation bodies 106 may be configured to prevent leaching or outgassing. In some examples, the agitation bodies 106 may be plastic, glass, metallic, or other materials. In various examples, the agitation bodies 106 may be specifically shaped such that the agitation bodies 106 homogenously disperse the unsuspended particles 105 when agitated. For example, the agitation bodies 106 may be spheres, oblate spheroids, prolate spheroids, cylinders, cones, cubes, cuboids, pyramids, prisms, or other geometric forms. The agitation bodies 106 may have a size ranging from about 1 mm to about 10 mm. In an example, the agitation bodies 106 may be 4 mm spheres.

In an aspect, the mechanical agitation system 100 may include about 1 to about 5 agitation bodies 106, about 5 to about 10 agitation bodies 106, about 10 to about 15 agitation bodies 106, about 15 to about 20 agitation bodies 106, about 20 to about 25 agitation bodies 106, about 25 to about 30 agitation bodies 106, about 30 to about 35 agitation bodies 106, about 35 to about 40 agitation bodies 106, about 40 to about 45 agitation bodies 106, about 45 to about 50 agitation bodies 106, about 50 to about 55 agitation bodies 106, about 55 to about 60 agitation bodies 106, about 60 to about 65 agitation bodies 106, about 65 to about 70 agitation bodies 106, about 70 to about 75 agitation bodies 106, about 75 to about 80 agitation bodies 106, about 80 to about 85 agitation bodies 106, about 85 to about 90 agitation bodies 106, about 90 to about 95 agitation bodies, about 95 to about 100 agitation bodies, or more. In another example, the mechanical agitation system 100 may include about 1 to about 100 agitation bodies 106, about 100 to about 200 agitation bodies 106, about 200 to about 300 agitation bodies 106, about 300 to about 400 agitation bodies 106, about 400 to about 500 agitation bodies 106, about 500 to about 600 agitation bodies 106, about 600 to about 700 agitation bodies 106, about 700 to about 800 agitation bodies 106, about 800 to about 900 agitation bodies 106, about 900 to about 1000 agitation bodies 106, or more.

The agitation bodies 106 may also have a hardness sufficient to prevent the formation of abrasion products. For example, the agitation bodies 106 may have a greater hardness than the cores of the particles. For example, if the core of the particle is a magnetite core, the hardness of the agitation bodies 106 may be at least about 5.5 Mohs. In another example, if the core of the particle is silica, the agitation bodies 106 may have a hardness of at least 7 Mohs. In some examples, the agitation bodies may have a hardness of about 5.5 Mohs to about 6 Mohs, about 6 Mohs to about 6.5 Mohs, about 6.5 Mohs to about 7 Mohs, about 7 Mohs to about 7.5 Mohs, about 7.5 Mohs to about 8 Mohs, about 8 Mohs to about 8.5 Mohs, about 8.5 Mohs to about 9 Mohs, about 9 Mohs to about 9.5 Mohs, or about 9.5 Mohs to about 10 Mohs. It will be appreciated that the hardness of the agitation bodies 106 may be selected for different applications depending on the hardness of the cores of the particles.

The agitation bodies 106 may have a different density from the carrier fluid 104. In some examples, the agitation bodies 106 may have a greater density than the carrier fluid 104. In another example, the agitation bodies 106 may have a lower density than the carrier fluid 104.

In an aspect, the mechanical agitation system 100 may include an agitation mechanism configured to agitate the one or more agitation bodies 106 within the syringe barrel 102 to disperse the unsuspended particles 105 in the carrier fluid 104. For example, the agitation mechanism may be a rocker plate 116 (e.g., reciprocating plate). The rocker plate 116 may be coupled to the barrel 102 of the syringe 108. The rocker plate 116 may include a rocker hub 117 and a rocker base 118. The rocker base 118 may be connected to a power source to provide power to the rocker plate 116. In some examples, the rocker plate 116 may be actuated by a motor. The rocker hub 117 may allow the rocker plate 116 to move. The rocker plate 116 may be configured to move such that the agitation bodies 106 are agitated. The rocker plate 116 may reciprocally move the barrel 102. Rocker plate motion 124 causes the agitation bodies 106 to move within the syringe barrel 102 such that the unsuspended particles 105 in the carrier fluid 104 are mechanically and homogenously dispersed. The rocker plate motion 124 may be forward-and-backward, side-to-side, up-and-down, circular, elliptical, nonlinear repetitive motion (e.g., tilting, swaying, metronome-like motion, pendulum motion, and/or other nonlinear repetitive motions), or combinations thereof.

In some aspects, the rocker plate 116 may have one or more frequencies and one or more amplitudes. In some examples, the frequency of the rocker plate 116 may be about 0.5 Hz to about 5 Hz, about 5 Hz to about 10 Hz, about 10 Hz to about 15 Hz, about 15 Hz to about 20 Hz, about 20 Hz to about 25 Hz, about 25 Hz to about 30 Hz, about 30 Hz to about 35 Hz, about 35 Hz to about 40 Hz, about 40 Hz to about 45 Hz, about 45 Hz to about 50 Hz, about 50 Hz to about 55 Hz, about 55 Hz to about 60 Hz, or more. The amplitude of the rocker plate 116 may depend on the type of motion. For example, the amplitude may be about 1 cm to about 2 cm, about 2 cm to about 3 cm, about 3 cm to about 4 cm, about 4 cm to about 5 cm, about 5 cm to about 6 cm, about 6 cm to about 7 cm, about 7 cm to about 8 cm, about 8 cm to about 9 cm, about 9 cm to about 10 cm, about 10 cm to about 15 cm, about 15 cm to about 20 cm, about 20 cm to about 25 cm, about 25 cm to about 30 cm, or more. In one example, when the rocker plate motion 124 is circular, the amplitude may be about 3 cm. In another example, when the rocker plate motion 124 is circular, the amplitude may be about 1 cm to about 5 cm. In a further example, the rocker plate motion 124 may be metronome-like or pendulum motion and the amplitude may be about 20 cm. In another example, the rocker plate motion 124 may be metronome-like or pendulum motion and the amplitude may be about 10 cm to about 30 cm.

In some aspects, a frequency of the rocker plate may be selected to control the homogenous dispersion of the unsuspended particles 105, the heating of the unsuspended particles 105 and carrier fluid 104, and/or a vibration of the system. For example, the frequency of the rocker plate 116 may directly affect the heating of the carrier fluid 104 and the unsuspended particles 105 for a given duration. The higher the frequency of the rocker plate 116, the greater the increase in heat within the syringe barrel 102 due to the agitation body motion 126 for a given duration.

In an aspect, the syringe 108 may have a syringe hub 112 at a proximal end of the syringe 108. The syringe hub 112 may be in fluid communication with the delivery conduit 114 (e.g., infusion tubing). In some examples, the delivery conduit 114 may have an inner diameter of about 0.1 mm to about 1 mm. In another example, the inner diameter of the delivery conduit may be about 0.1 mm to about 1 mm, about 1 mm to about 2 mm, about 2 mm to about 3 mm, about 3 mm to about 4 mm, or about 4 mm to about 5 mm. The syringe hub 112 may have a diameter less than a minimum size of the agitation bodies 106, thereby blocking the agitation bodies 106 from entering the delivery conduit 114. The agitation bodies 106 may remain in the barrel 102 throughout the infusion process.

In some aspects, motion of the agitation bodies 106 may heat the carrier fluid 104 and/or the unsuspended particles 105 within the syringe barrel 102. The heat generated may be less than the body temperature of a patient. In some examples, the unsuspended particles 105 and the carrier fluid 104 are delivered to a patient at a temperature less than the body temperature of the patient.

In some examples, the mechanical agitation system 100 may further include a cooling mechanism. The cooling mechanism may be operable to cool the unsuspended particles 105 and carrier fluid 104 within the syringe barrel 102. In some examples, the cooling mechanism may be a fan operable to provide cooling air to the exterior of the syringe barrel 102. In some examples, the cooling air may be room temperature or below room temperature. In other examples, the cooling mechanism may be a chemical cooling mechanism. Cooling bodies may be placed within the barrel 102 of the syringe 108 such that when the rocker plate 116 begins moving the agitation bodies 106, the cooling bodies are activated providing a cooling effect on the carrier fluid 104 and the unsuspended particles 105. In further examples, the syringe barrel 102 may be wrapped in a cooling jacket to allow energy to transfer out of the syringe, thereby cooling the carrier fluid 104 and the unsuspended particles 105 within the syringe barrel 102. The temperature of the carrier fluid 104 and unsuspended particles 105 is about 4 degrees C. to about 37 degrees C. In some examples, the unsuspended particles 105 and the carrier fluid 104 are delivered to a patient at a temperature less than the body temperature of the patient. In some examples, the unsuspended particles 105 and the carrier fluid 104 may be administered at a temperature around room temperature. It will be appreciated that the cooling mechanism may be used with any of the systems described herein.

FIGS. 1A-1D illustrate the motion of the rocker plate 116. As illustrated in FIG. 1A, the rocker plate 116 may begin in a first position 101. The rocker plate 116 and rocker hub 117, may begin rocker plate motion 124 to a second position 121, as illustrated, for example, in FIG. 1B. In this example, the rocker plate 116 moves in a side-to-side or backward-and-forward motion. When the rocker plate 116 begins rocker plate motion 124 in a backward direction (e.g., towards the syringe plunger 103), the agitation bodies 106 may move forward (e.g., towards the syringe hub 112), as illustrated as agitation body motion 126. At a third position 131, as illustrated in FIG. 1C, the rocker plate motion 124 is in a forward direction, causing agitation body motion 126 in a backward direction (e.g., towards the syringe plunger 103). At a fourth position 141, as illustrated in FIG. 1D, the rocker plate motion 124 may be in a backward direction (e.g., towards the syringe plunger 103) causing agitation body motion 126 in a forward direction (e.g., towards the syringe hub 112). By creating rocker plate motion 124 and agitation body motion 126, the unsuspended particles 105 become mechanically, homogenously dispersed within the carrier fluid 104.

In an aspect, the syringe plunger 103 may be operable to push the unsuspended particles 105 and the carrier fluid 104 into the delivery conduit 114 by providing a force 122 to the plunger 103 (e.g., compressing the plunger 103), thereby delivering the unsuspended particles 105 to a patient. In some examples, rocker plate motion 124 and agitation body motion 126 may occur for a warm-up period before compressing the syringe plunger 103 to begin delivery. For example, the warm-up period may be about 1 second to about 5 second, about 5 seconds to about 10 seconds, about 10 seconds to about 15 seconds, about 15 seconds to about 20 seconds, about 20 seconds to about 25 seconds, about 25 seconds to about 30 seconds, about 30 seconds to about 35 seconds, about 35 seconds to about 40 seconds, about 40 seconds to about 45 seconds, about 45 seconds to about 50 seconds, about 50 seconds to about 55 seconds, about 55 seconds to about 1 minute, about 1 minute to about 2 minutes, about 2 minutes to about 3 minutes, about 3 minutes to about 4 minutes, or about 4 minutes to about 5 minutes. In other examples, no warm-up period is necessary and the syringe plunger 103 and the rocker plate 116 may be activated at the same time.

In some aspects, the inner diameter of the delivery conduit 114, viscosity of the carrier fluid 104, density of the unsuspended particles 105, and hydrodynamic diameter of the unsuspended particles 105 may determine the critical flow velocity to keep the unsuspended particles 105 entrained in the carrier fluid 104. Once the critical flow velocity to keep the unsuspended particles entrained in the carrier fluid 104 is determined, the critical flow velocity may be multiplied by the inner diameter of the delivery conduit 114 to determine the necessary flow rate of the carrier fluid 104 to entrain the unsuspended particles. Using this determined flow rate, the pressure, and therefore the force 122 provided to the syringe plunger 103, necessary to achieve the required flow rate of the carrier fluid 104 to entrain the unsuspended particles 105 may be determined. In some examples, the carrier fluid flow rate is sufficient to entrain the unsuspended particles 105 in the carrier fluid 104, thereby maintaining homogenous dispersion of the unsuspended particles 105. By entraining the unsuspended particles 105 it is ensured that the unsuspended particles 105 do not fall out of the carrier fluid and collect on the side wall of the tubing. It will be appreciated that the critical velocity of the carrier fluid 104 for entrainment and carrier fluid flow rate determination applies to all of the systems and methods disclosed herein.

In some examples, the critical velocity may be 10 cm/s for entraining the unsuspended particles 105 in saline. In this example, when the critical velocity to entrain the unsuspended particles 105 in saline is 10 cm/s, the required flow rate of the carrier fluid 104 is 1.2 mL/min for a 0.5 mm inner diameter delivery conduit 114. In another example, the critical velocity may be 3 cm/s for entraining the unsuspended particles 105 in water. In this example, the flow rate of the carrier fluid 104 to entrain the unsuspended particles 105 may be about 4 mL/min for a 1.7 mm inner diameter delivery conduit. In another example a velocity of the carrier fluid 104 may be 6 cm/s. In this example, the flow rate of the carrier fluid 104 to entrain the unsuspended particles 105 may be about 0.66 mL/min for a 0.5 mm inner diameter delivery conduit. It will be appreciated that other flow rates may be chosen depending on the critical velocity necessary to entrain the unsuspended particles 105 and the inner diameter of the delivery conduit 114.

In other examples, the unsuspended particles 105 may be delivered at desired heterogenous rates. Heterogenous rates may be achieved by dynamically adjusting the concentration of unsuspended particles 105 in the at least one reservoir (e.g., syringe barrel 102) by continuously adding more carrier fluid 104 or by dynamically changing the volumetric flow rate.

In another aspect, the unsuspended particles 105 may be mechanically, homogenously dispersed in a second reservoir. In this example, the unsuspended particles 105 and agitation bodies 106 are not placed in the barrel 102 of the syringe 108, but rather are held in the second reservoir. The rocker plate 116, rocker hub 117, and rocker base 118 may be coupled to the second reservoir. The rocker plate 116 may cause motion of the agitation bodies 106, and therefore homogenous dispersion of the unsuspended particles 105, in the second reservoir. The syringe hub 112 may be in fluid communication with the second reservoir via a tube (e.g., conduit). By compressing the plunger 103, the carrier fluid 104 may be expelled from the syringe hub 112 and into the tube connecting the syringe 108 to the second reservoir. The unsuspended particles 105 are then infused with the carrier fluid 104 in the second reservoir. The carrier fluid 104 enters the second reservoir at a sufficient flow rate such that the mechanically mixed unsuspended particles 105 are infused at a desired homogeneity. In other examples, the unsuspended particles 105 may be entrained in the carrier fluid 104 at desired heterogenous rates.

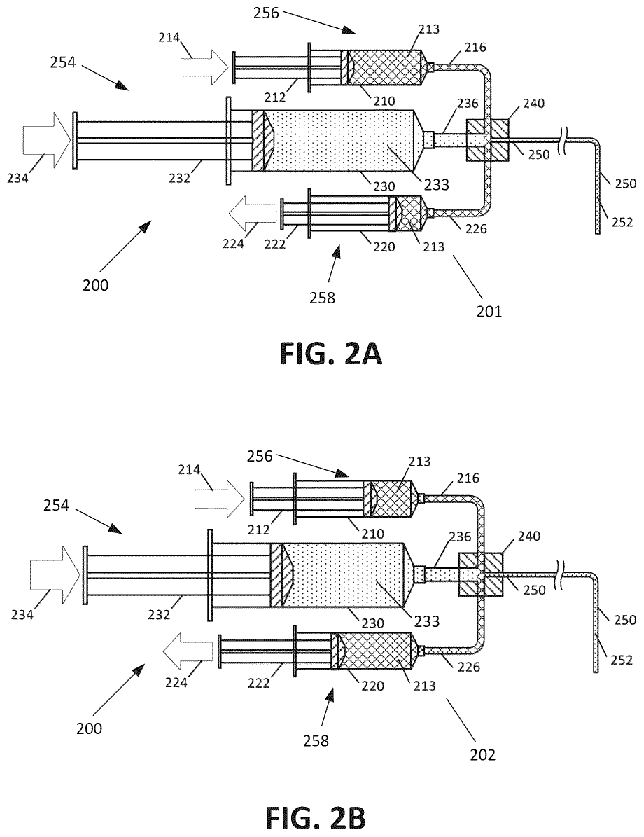

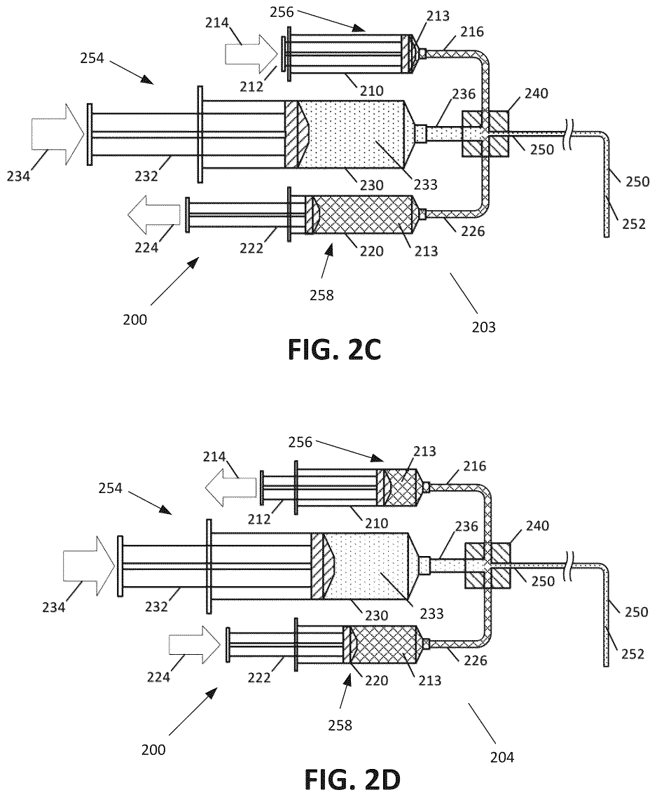

FIGS. 2A-2D illustrate a mechanical agitation system 200 in another example. The mechanical agitation system 200 may include an agitation mechanism (e.g., cycling mechanism) comprising a first syringe 256 (e.g., agitation syringe), a second syringe 258 (e.g., agitation syringe), and a third syringe 254 (e.g., infusion syringe). The third syringe may have a plunger 232 and a barrel 230 (e.g., third reservoir). A carrier fluid 233 (i.e., infusion fluid to entrain the unsuspended particles) may be held within the barrel 230 of the third syringe 254. A hub of the third syringe 254 may be in fluid communication with a first manifold tubing 236. The first manifold tubing may be operable to provide the carrier fluid 233 to a manifold 240. The first syringe 256, second syringe 258, and third syringe 254 may be in fluid communication with the manifold.

In some aspects, the first syringe 256 may have a plunger 212 and a barrel 210 (e.g., first reservoir). Unsuspended particles 213 may be held within the barrel 210 of the first syringe 256. The first syringe 256 may have a hub in fluid communication with a second manifold tubing 216. The second manifold tubing 216 may be operable to provide the unsuspended particles 213 to the manifold 240. In some examples, the second manifold tubing 216 may also be operable to provide unsuspended particles 213 to the first syringe 256 from the manifold 240.

In an aspect, the second syringe 258 may have a plunger 222 and a barrel 220 (e.g., second reservoir). Unsuspended particles 213 may be held within the barrel 220 of the second syringe 258. The second syringe 258 may have a hub in fluid communication with a third manifold tubing 226. The third manifold tubing 226 may be operable to provide the unsuspended particles 213 to the manifold. In some examples, the third manifold tubing 226 may also be operable to provide unsuspended particles 213 to the second syringe 258 from the manifold 240.

In some aspects, the manifold 240 may have four inlets/outlets. The first inlet of the manifold 240 may be in fluid communication with the first manifold tubing 236. The first inlet may only allow flow in one direction (e.g., from the third syringe 254 to the manifold 240). The first inlet may be operable to receive the carrier fluid 233 from the third syringe 254. The second inlet/outlet of the manifold 240 may be in fluid communication with the second manifold tubing 216. The second inlet/outlet of the manifold may allow flow in two directions. The second inlet/outlet may receive unsuspended particles 213 from the first syringe 256 and also provide unsuspended particles 213 to the first syringe 256 from the manifold 240. The third inlet/outlet may be in fluid communication with the third manifold tubing 226. The third inlet/outlet of the manifold 240 may allow flow in two directions. The third inlet/outlet may be operable to receive unsuspended particles 213 from the second syringe 258 and provide unsuspended particles 213 to the second syringe 258. The fourth outlet of the manifold 240 may provide the carrier fluid 233 and the unsuspended particles 213 to the delivery conduit 250. As illustrated in FIGS. 2A-2D, the carrier fluid 233 may entrain the unsuspended particles 213 in the manifold 240 and deliver the entrained unsuspended particles 252 (e.g., homogenous mixture) to a patient or infusion apparatus.

As illustrated in FIGS. 2A-2D, the first syringe 256 and second syringe 258 may cycle the unsuspended particles 213 back and forth through the manifold 240. By cycling the unsuspended particles back and forth between the first syringe 256 and the second syringe 258, the unsuspended particles 213 may be homogenously distributed due to the kinetic energy of the unsuspended particles 213. In some examples, the unsuspended particles 213 may be cycled back and forth for a warm-up period before the third syringe 254 begins providing the carrier fluid 233 to the manifold 240. For example, the warm-up period may be about 1 second to about 5 second, about 5 seconds to about 10 seconds, about 10 seconds to about 15 seconds, about 15 seconds to about 20 seconds, about 20 seconds to about 25 seconds, about 25 seconds to about 30 seconds, about 30 seconds to about 35 seconds, about 35 seconds to about 40 seconds, about 40 seconds to about 45 seconds, about 45 seconds to about 50 seconds, about 50 seconds to about 55 seconds, about 55 seconds to about 1 minute, about 1 minute to about 2 minutes, about 2 minutes to about 3 minutes, about 3 minutes to about 4 minutes, or about 4 minutes to about 5 minutes.

After the optional warm-up period, the plunger 232 of the third syringe 254 may begin being compressed, as shown by third syringe plunger motion 234 in FIGS. 2A-2D. Cycling the unsuspended particles 213 between the first syringe 256 and the second syringe 258 includes compressing (e.g., pushing the plunger towards the syringe hub) and decompressing (e.g., pulling the plunger away from the syringe hub) the plunger 212 of the first syringe 256 and the plunger 222 of the second syringe 258. As illustrated in FIG. 2A, the cycling of the unsuspended particles 213 may begin at a first position 201. When the cycling of the unsuspended particles 213 begins the plunger 212 of the first syringe 256 is compressed, as illustrated by first syringe plunger motion 214, thereby decreasing the volume of unsuspended particles 213 in the first syringe 256. The plunger 222 of the second syringe 258 is decompressed, as illustrated by the second syringe plunger motion 224. The first syringe plunger motion 214 causes the unsuspended particles 213 to be ejected from the first syringe 256 into the second manifold tubing 216 and into the manifold 240. Some of the unsuspended particles 213 from the first syringe 256 are entrained in the carrier fluid 233 provided by compressing the plunger 232 of the third syringe 254. The entrained unsuspended particles 252 (e.g., homogenous mixture) flow through the delivery conduit 250 to the patient or infusion apparatus. The plunger 222 of the second syringe is decompressed allowing unsuspended particles 213 that have not been entrained in the carrier fluid 233 to fill the barrel 220 of the second syringe 258 through the third manifold tubing 226.

In some aspects, the inner diameter of the delivery conduit 250, viscosity of the carrier fluid 233, density of the unsuspended particles 213, and hydrodynamic diameter of the unsuspended particles 213 may be used to determine the critical flow velocity to keep the unsuspended particles 213 entrained in the carrier fluid 233. Once the critical flow velocity to keep the unsuspended particles entrained in the carrier fluid 104 is determined, the critical flow velocity may be multiplied by the inner diameter of the delivery conduit 250 to determine the necessary flow rate of the carrier fluid 233 to entrain the unsuspended particles. Using this determined flow rate, the pressure, and therefore the force 234 provided to the plunger 232 of the third syringe 254, necessary to achieve the required flow rate of the carrier fluid 233 to entrain the unsuspended particles 213 may be determined. In some examples, the carrier fluid flow rate is sufficient to entrain the unsuspended particles 213 in the carrier fluid 233, thereby maintaining homogenous dispersion of the unsuspended particles 213. By entraining the unsuspended particles 213, it is ensured that the unsuspended particles 213 do not fall out of the carrier fluid and collect on the side wall of the tubing. It will be appreciated that the critical velocity of the carrier fluid 233 for entrainment and carrier fluid flow rate determination applies to all of the systems and methods disclosed herein.

In some examples, the critical velocity may be 10 cm/s for entraining the unsuspended particles 213 in saline. In this example, when the critical velocity to entrain the unsuspended particles 213 in saline is 10 cm/s the required flow rate of the carrier fluid 104 is 1.2 mL/min for a 0.5 mm inner diameter delivery conduit 250. In another example, the critical velocity may be 3 cm/s for entraining the unsuspended particles 105 in water. In this example, the flow rate of the carrier fluid 104 to entrain the unsuspended particles 105 may be about 4 mL/min for a 1.7 mm inner diameter delivery conduit. In another example a velocity of the carrier fluid 233 may be 6 cm/s. In this example, the flow rate of the carrier fluid 213 to entrain the unsuspended particles 213 may be about 0.66 mL/min for a 0.5 mm inner diameter delivery conduit. It will be appreciated that other flow rates may be chosen depending on the critical velocity necessary to entrain the unsuspended particles 213 and the inner diameter of the delivery conduit 250.

As illustrated in FIGS. 2B-2C, the first syringe plunger motion 214 may continue until all of the unsuspended particles 213 have been expelled from the first syringe 256. FIG. 2B illustrates a second position 202 of the cycle. In the second position 202, the first syringe plunger motion 214 continues to expel unsuspended particles 213 from the first syringe 256. In the second position 202, the second syringe plunger motion 224 continues in a backward direction, thereby allowing the unsuspended particles 213 that are not entrained in the carrier fluid to fill the barrel 220 of the second syringe 258. In the third position 203, as illustrated in FIG. 2C, all of the unsuspended particles 213 have been expelled from the first syringe 256. At the third position 203, all of the unsuspended particles 213 that have not been entrained in the carrier fluid 233 are located in the barrel 220 of the second syringe 258.

As illustrated in FIG. 2D, once all of the unsuspended particles 213 have been expelled from the barrel 210 of the first syringe 256, the first syringe plunger motion 214 may be reversed such that the plunger 212 of the first syringe 256 begins decompressing. Similarly, the direction of the second syringe plunger motion 224 may be reversed, such that the plunger 222 of the second syringe 258 begins compressing. The volume of the unsuspended particles 213 will be less than the original volume of unsuspended particles since some of the unsuspended particles 213 have been entrained in the carrier fluid 233. The cycle may then continue in substantially the same way as described above. For example, the second syringe plunger motion 224 may continue in a forward direction until all of the unsuspended particles 213 are expelled from the barrel 220 of the second syringe 258. The first syringe plunger motion 214 may continue in a backward direction allowing a volume of unsuspended particles 213, that are not entrained in the carrier fluid 233 while in the manifold 240, to fill the barrel 210 of the first syringe 256. The cycling of unsuspended particles 213 may continue in substantially the same way until all of the unsuspended particles 213 have been entrained in the carrier fluid 233.

In some aspects, the third syringe plunger motion 234 may be configured to ensure a sufficient flow rate of the carrier fluid 233 to allow for entrainment of the unsuspended particles 213. In some examples, the combination of the third syringe plunger motion 234 (e.g., compression speed) and the diameter of the first manifold tubing 236 may provide an adequate flow rate of the carrier fluid 233 to entrain at least some of the unsuspended particles 213 flowing through the manifold 240 from the first syringe 256 and the second syringe 258. The pressure differential between the carrier fluid 233 flow and the unsuspended particles 213 flow allows some of the particles to be entrained in the carrier fluid 233 in the manifold 240. This pressure differential allows a constant delivery of unsuspended particles 213 to the delivery conduit 250.

In some examples, the third syringe 254, first syringe 256, and second syringe 258 may be motorized and controlled by at least one processor. The syringes may be actuated (e.g., compressed or decompressed) automatically by a processor. The motorized syringes may then provide a constant flow of entrained unsuspended particles 252 (e.g., homogenous mixture) to the patient or infusion apparatus.

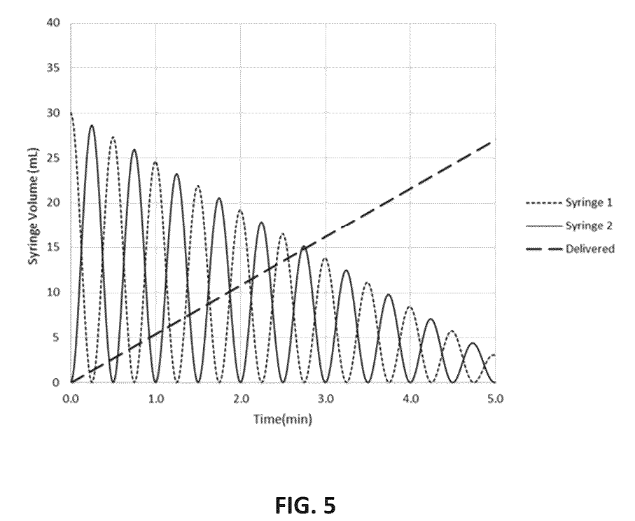

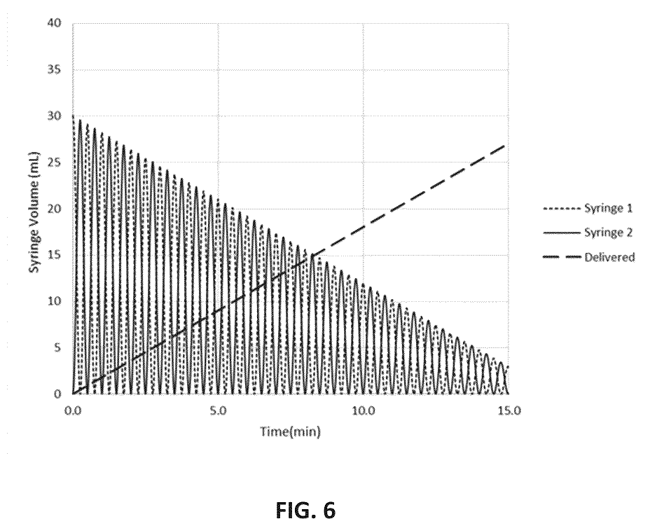

FIGS. 5-6 illustrate constant delivery rates of the entrained unsuspended particles 252 (e.g., homogenous mixture) based on the volumes of the first syringe and second syringe. The first syringe volume 504 begins completely filled with unsuspended particles 213. The first syringe volume may be about 1 mL to about 10 mL, about 10 mL to about 20 mL, about 20 mL to about 30 mL, about 30 mL to about 40 mL, about 40 mL to about 50 mL, about 50 mL to about 60 mL, about 60 mL to about 70 mL, about 70 mL to about 80 mL, about 80 mL to about 90 mL, or about 90 mL to about 100 mL. In another example, the first syringe volume may be about 5 mL to about 100 mL. In the example illustrated in FIG. 5, the first syringe volume is 30 mL. In other examples, different volumes of unsuspended particles 213 may be used. The volume of the second syringe may be the same as the volume of the first syringe. In some examples, the third syringe volume (e.g., the volume of the carrier fluid) may be about 5 mL to about 10 mL, about 10 mL to about 20 mL, about 20 mL to about 30 mL, about 30 mL to about 40 mL, about 40 mL to about 50 mL, about 50 mL to about 60 mL, about 60 mL to about 70 mL, about 70 mL to about 80 mL, about 80 mL to about 90 mL, about 90 mL to about 100 mL, about 100 mL to about 110 mL, about 110 mL to about 120 mL, about 120 mL to about 130 mL, about 130 mL to about 140 mL, about 140 mL to about 150 mL, about 150 mL to about 160 mL, about 160 mL to about 170 mL, about 170 mL to about 180 mL, about 180 mL to about 190 mL, about 190 mL to about 200 mL, or more.

As the plunger of the first syringe is compressed, as described above, the first syringe volume 504 decreases while the second syringe volume 502 increases. When the plunger of the first syringe begins to decompress and the plunger of the second syringe begins to compress, the second syringe volume 502 decreases and the first syringe volume 504 increases. As illustrated in FIGS. 5 and 6, the unsuspended particles delivered 500 corresponds to the decrease in total volume between the first syringe and the second syringe. This decrease in total volume is a result of some of the unsuspended particles becoming entrained in the carrier fluid. In some examples, the cycle period (e.g., the period between a syringe having a full volume and zero volume) may be about 5 seconds to about 10 seconds, about 10 seconds to about 15 seconds, about 15 seconds to about 20 seconds, about 20 seconds to about 25 seconds, about 25 seconds to about 30 seconds, about 30 seconds to about 35 seconds, about 35 seconds to about 40 seconds, about 40 seconds to about 45 seconds, about 45 seconds to about 50 seconds, about 50 seconds to about 55 seconds, or about 55 seconds to about 1 minute.

In some aspects, a processor may be used to determine the necessary critical velocity of the carrier fluid to entrain the unsuspended particles for infusion, as described above. In an example, the processor may be in communication with a display to display information related to the administration of unsuspended particles. The display may be a visual display operable to receive an input from the user of various parameters (e.g., particle concentration, volume of unsuspended particles to be administered, unsuspended particle mass, density of the particles, density of the carrier fluid, viscosity of the carrier fluid, diameter of the delivery conduit, hydrodynamic diameter of the particles, etc.). The processor may then provide the necessary pressure to the carrier fluid to achieve the critical velocity to entrain the particles.

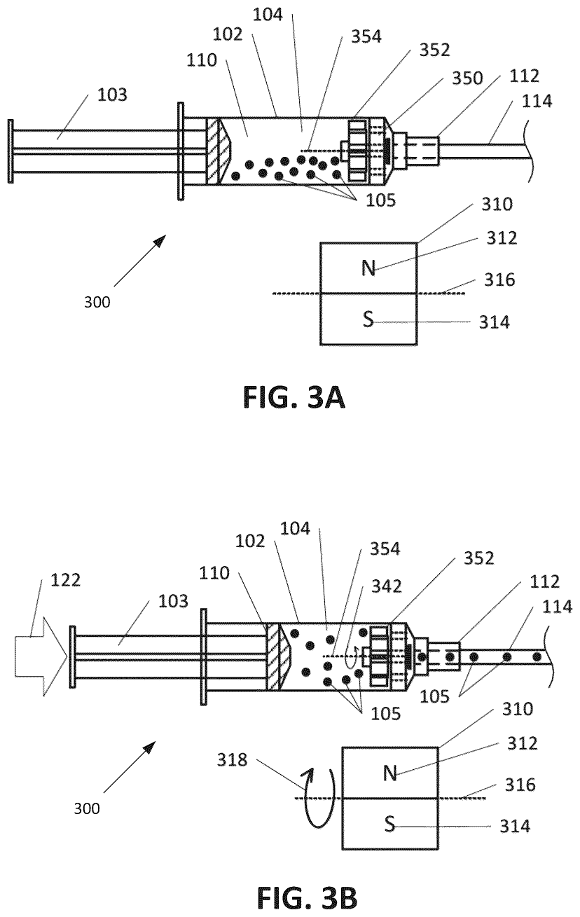

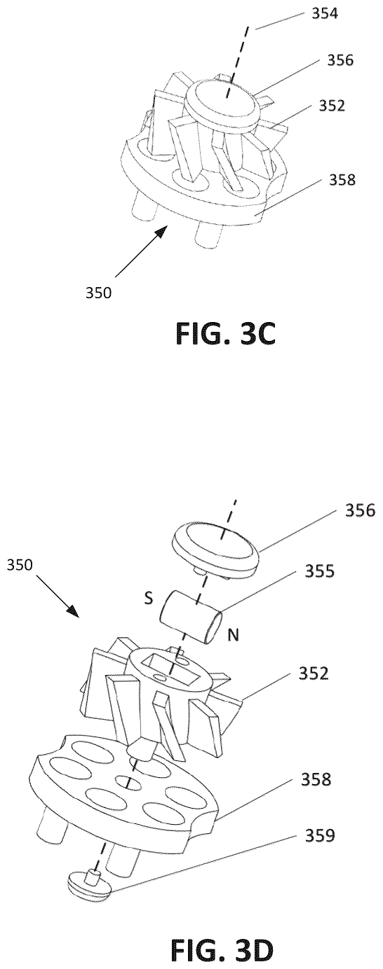

As illustrated in FIG. 3A-B, the system for controlled administration of unsuspended particles may be a magnetic mixer system 300. The magnetic mixer system 300 may include a magnetic mixer assembly 350 (e.g., magnetic rotating element), a syringe having a barrel 102 (e.g., reservoir) and a plunger 103 (e.g., pressure source), and an external magnet subassembly 310. The magnetic mixer system 300 may further include a delivery conduit 114 in fluid communication with the syringe hub 112. The magnetic mixer assembly 350 may be an agitation mechanism. The syringe barrel 102 may be loaded with a carrier fluid 104 and the unsuspended particles 105.

As illustrated in FIGS. 3C-3D, the magnetic mixer assembly 350 may include a base 358, a magnetic mixing fan 352, a mixing fan magnet 355, a first retaining cap 356 and a second retaining cap 359. The second retaining cap 359 may keep the magnetic mixing fan 352 connected to the base 358. The first retaining cap 356 may be configured to keep the mixing fan magnet 355 within the recess of the magnetic mixing fan 352. FIG. 3C illustrates the axis of rotation 354 of the magnetic mixing fan 352.

In some aspects, the magnetic mixing fan 352 may have various shapes. For example, the magnetic mixing fan 352 may have 1, 2, 3, 4, 5, 6, 7, 8, 9, 10, or more blades. In some examples, the blades may be evenly spaced from one another. In another example, the blades may not be evenly spaced from another (e.g., two blades may have less space between them than between a third blade). In some examples, the blades may have any shape profile known in the art (e.g., curved, flat, rounded, etc.). In some examples, the blades may extend from a center of magnetic mixing fan 352 at an angle. In an example, each blade may extend from the center of the magnetic mixing fan 352 at an angle of about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, or about 80 degrees to about 90 degrees in any direction.

As illustrated in FIGS. 3A-3B, the magnetic mixer system 300 may include an external magnet subassembly 310. The external magnet subassembly 310 may include an external magnet having a north pole 312 and a south pole 314. The external magnet may rotate around external magnet rotation axis 316. The external magnet subassembly 310 may generate a sufficient magnetic field to cause the magnetic mixing fan 352 to rotate. The external magnet subassembly 310 may be located near an exterior of the syringe barrel 102. In some examples, the magnetic field may be about 25 millitesla (mT) to about 50 mT, about 50 mT to about 75 mT, about 75 mT to about 100 mT, about 100 mT to about 125 mT, about 125 mT to about 150 mT, about 150 mT to about 175 mT, about 175 mT to about 200 mT, about 200 mT to about 225 mT, about 225 mT to about 250 mT, about 250 mT to about 275 mT, about 275 mT to about 300 mT, about 300 mT to about 325 mT, about 325 mT to about 350 mT, about 350 mT to about 375 mT, about 375 mT to about 400 mT, about 400 mT to about 425 mT, about 425 mT to about 450 mT, about 450 mT to about 475 mT, about 475 mT to about 500 mT, or more.

The magnetic mixer assembly 350 may be attached to or placed in an interior of the barrel 102 of the syringe. In some examples, the magnetic mixer assembly 350 may be fixed to the distal portion (e.g., near the syringe hub 112) of the syringe. In other examples, the magnetic mixer assembly 350 may be fixed to the proximal portion (e.g., on the end of the plunger 103 within the barrel 102) of the syringe. In further examples, the magnetic mixer assembly 350 may have the same diameter as the barrel 102. In an example, the magnetic mixer assembly 350 may fit within the barrel 102 using a friction fit. In another example, the magnetic mixer assembly 350 may be operable to slide back and forth within the barrel 102. In some examples, more than one magnetic mixer assembly may be placed within the barrel 102 of the syringe. For example, a magnetic mixer assembly 350 may be attached to the distal portion of the syringe and another magnetic mixer assembly 350 may be attached to a proximal portion of the syringe.

As illustrated in FIG. 3B, the external magnet subassembly 310 may rotate clockwise as illustrated by rotation arrow 318. Rotation of the external magnet subassembly 310 may cause the magnetic mixing fan 352 to rotate. The rotation of the magnetic mixing fan 352 may cause fluidic mixing of unsuspended particles 105. Fluidic mixing of the unsuspended particles causes homogenous dispersion of the unsuspended particles 105. In some examples, the rate of change in the magnetic field provided by the rotation of the external magnet subassembly 310 may determine the rate of fluidic mixing. In some examples, the rate of change in the magnetic field may be about 1 Hz to about 2 Hz, about 2 Hz to about 3 Hz, about 3 Hz to about 4 Hz, about 4 Hz to about 5 Hz, about 5 Hz to about 6 Hz, about 6 Hz to about 7 Hz, about 7 Hz to about 8 Hz, about 8 Hz to about 9 Hz, about 9 Hz to about 10 Hz. In another example, the rate of change of the magnetic field may be about 1 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, or more. Increasing the speed of the rotation of the external magnet subassembly 310 increases the rate of change of the magnetic field, and thereby the speed of the fluidic mixing. Decreasing the speed of the rotation of the external magnet subassembly 310 decreases the rate of change of the magnetic field, and thereby the speed of the fluidic mixing.

In another aspect, the external magnet subassembly 310 may be an electrical coil wrapped around the syringe barrel 102. The electrical coil may be operable to provide the magnetic field to rotate the magnetic mixing fan 352. In another example, the electrical coil may be operable to replace both the external magnet subassembly 310 and the magnetic mixer assembly 352. The electrical coil may be operable to provide varying magnetic fields to the unsuspended particles 105. When the unsuspended particles 105 are magnetic particles, the magnetic field provided by the electrical coil may be operable to homogenously disperse the unsuspended particles 105.

In some aspects, the unsuspended particles 105 may be agitated, and thereby homogenously dispersed, prior to infusing the unsuspended particles 105 and carrier fluid 104 into the delivery conduit 114. This period is referred to as an optional warm-up period. For example, the warm-up period may be about 1 second to about 5 second, about 5 seconds to about 10 seconds, about 10 seconds to about 15 seconds, about 15 seconds to about 20 seconds, about 20 seconds to about 25 seconds, about 25 seconds to about 30 seconds, about 30 seconds to about 35 seconds, about 35 seconds to about 40 seconds, about 40 seconds to about 45 seconds, about 45 seconds to about 50 seconds, about 50 seconds to about 55 seconds, about 55 seconds to about 1 minute, about 1 minute to about 2 minutes, about 2 minutes to about 3 minutes, about 3 minutes to about 4 minutes, or about 4 minutes to about 5 minutes.

While the unsuspended particles 105 are being homogenously dispersed by the magnetic mixer assembly 350, the unsuspended particles 105 may be delivered to the delivery conduit 114 by compressing (e.g., providing a force to) the plunger 103 of the syringe, as illustrated by force 122. In some aspects, the inner diameter of the delivery conduit 114, viscosity of the carrier fluid 104, density of the unsuspended particles 105, and hydrodynamic diameter of the unsuspended particles 105 may determine the critical flow velocity to keep the unsuspended particles 105 entrained in the carrier fluid. Once the critical flow velocity to keep the unsuspended particles entrained in the carrier fluid 104 is determined, the critical flow velocity may be multiplied by the inner diameter of the delivery conduit 114 to determine the necessary flow rate of the carrier fluid 104 to entrain the unsuspended particles. Using this determined flow rate, the pressure, and therefore the force 122 provided to the syringe plunger 103, necessary to achieve the required flow rate of the carrier fluid 104 to entrain the unsuspended particles 105 may be determined. In some examples, the carrier flow rate is sufficient to entrain the unsuspended particles 105 in the carrier fluid 104, thereby maintaining homogenous dispersion of the unsuspended particles 105. By entraining the unsuspended particles 105 it is ensured that the unsuspended particles 105 do not fall out of the carrier fluid and collect on the side wall of the tubing. It will be appreciated that the critical velocity of the carrier fluid 104 for entrainment and carrier fluid flow rate determination applies to all of the systems and methods disclosed herein.

In some examples, the critical velocity may be 10 cm/s for entraining the unsuspended particles 105 in saline. In this example, when the critical velocity to entrain the unsuspended particles 105 in saline is 10 cm/s the required flow rate of the carrier fluid 104 is 1.2 mL/min for a 0.5 mm inner diameter delivery conduit 114. In another example, the critical velocity may be 3 cm/s for entraining the unsuspended particles 105 in water. In this example, the flow rate of the carrier fluid 104 to entrain the unsuspended particles 105 may be about 4 mL/min for a 1.7 mm inner diameter delivery conduit. In another example a velocity of the carrier fluid 104 may be 6 cm/s. In this example, the flow rate of the carrier fluid 104 to entrain the unsuspended particles 105 may be about 0.66 mL/min for a 0.5 mm inner diameter delivery conduit. It will be appreciated that other flow rates may be chosen depending on the critical velocity necessary to entrain the unsuspended particles 105 and the inner diameter of the delivery conduit 114.

In another aspect, the unsuspended particles 105 may be homogenously dispersed in a second reservoir. In this example, the unsuspended particles 105 are not placed in the barrel 102 of the syringe 108, but rather are held in the second reservoir. The magnetic mixer assembly 350 may be located (e.g., attached or placed) in the second reservoir. The magnetic mixer assembly 350 may cause fluidic mixing of the unsuspended particles 105, and therefore homogenous dispersion of the unsuspended particles 105, in the second reservoir. The syringe hub 112 may be connected to the second reservoir via a tube. By compressing the plunger 103, the carrier fluid 104 may be expelled from the syringe hub 112 and into the tube connecting the syringe to the second reservoir. The unsuspended particles 105 are then infused with the carrier fluid 104 in the second reservoir. The carrier fluid 104 enters the second reservoir at a sufficient flow rate such that the fluidically mixed unsuspended particles 105 are infused at a desired homogeneity. In other examples, the unsuspended particles 105 may be entrained in the carrier fluid 104 at desired heterogenous rates. The entrained unsuspended particles 105 may then be delivered to a patient via the delivery conduit 114, which is in fluid communication with the second reservoir.

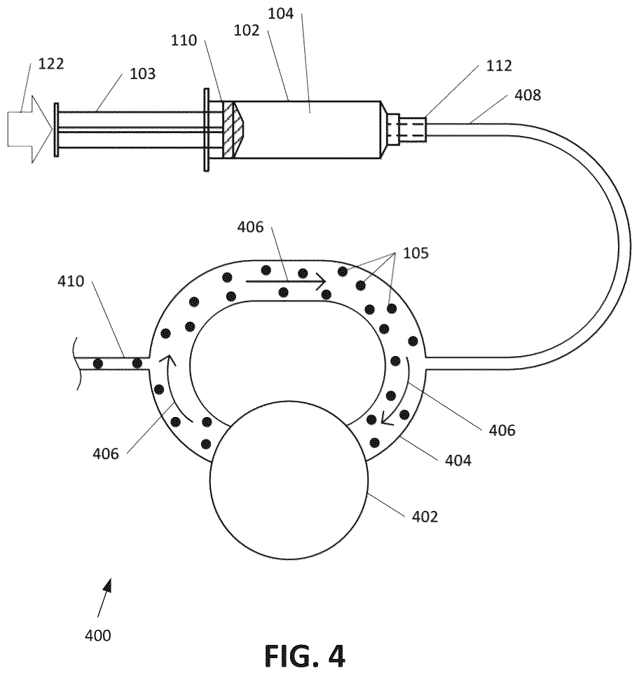

As illustrated in FIG. 4, the system for controlled administration of unsuspended particles may be a peristaltic agitation system 400. The peristaltic agitation system may include a syringe having a barrel 102 (e.g., carrier fluid reservoir) and a plunger 103 (e.g., pressure source). The barrel 102 may contain a carrier fluid 104. The carrier fluid 104 may be expelled from the syringe hub 112 to a deliver the carrier fluid 104 to a peristaltic reservoir 404 (e.g., fluid loop). The peristaltic agitation system 400 may include a peristaltic pump 402. The barrel 102 (e.g., carrier fluid reservoir) may be in fluid communication with the peristaltic reservoir 404 (e.g., fluid loop) on one end of the peristaltic reservoir 404 and the delivery conduit 410 may be in fluid communication with the peristaltic reservoir 404 on an opposite end.

As illustrated in FIG. 4, the unsuspended particles 105 may be loaded into the peristaltic reservoir 404. The peristaltic pump 402 may provide motion of the unsuspended particles 105 in the peristaltic reservoir 404 in the direction indicated by arrow 406. The motion of the unsuspended particles 105 in the peristaltic reservoir 404 may homogenously disperse the unsuspended particles 105 in the peristaltic reservoir 404.

The carrier fluid 104 may be provided to the peristaltic reservoir 404 by compressing (e.g., providing a force to) the plunger 103. The carrier fluid 104 may travel to the peristaltic reservoir 404 via peristaltic reservoir inlet tubing 408. Once the carrier fluid 104 arrives in the peristaltic reservoir 404, the unsuspended particles 105 may be entrained in the carrier fluid. The entrained unsuspended particles 105 and carrier fluid may then travel to a patient or infusion apparatus via delivery conduit 410, which is in fluid communication with the peristaltic reservoir 404.

In some aspects, the inner diameter of the delivery conduit 410, viscosity of the carrier fluid 104, density of the unsuspended particles 105, and hydrodynamic diameter of the unsuspended particles 105 may determine the critical flow velocity to keep the unsuspended particles 105 entrained in the carrier fluid. Once the critical flow velocity to keep the unsuspended particles entrained in the carrier fluid 104 is determined, the critical flow velocity may be multiplied by the inner diameter of the delivery conduit 410 to determine the necessary flow rate of the carrier fluid 104 to entrain the unsuspended particles. Using this determined flow rate, the pressure, and therefore the force 122 provided to the syringe plunger 103 and/or the pressure provided by the peristaltic pump 402, necessary to achieve the required flow rate of the carrier fluid 104 to entrain the unsuspended particles 105 may be determined. In some examples, the carrier flow rate is sufficient to entrain the unsuspended particles 105 in the carrier fluid 104, thereby maintaining homogenous dispersion of the unsuspended particles 105. By entraining the unsuspended particles 105 it is ensured that the unsuspended particles 105 do not fall out of the carrier fluid and collect on the side wall of the tubing. It will be appreciated that the critical velocity of the carrier fluid 104 for entrainment and carrier fluid flow rate determination applies to all of the systems and methods disclosed herein.

In some examples, the critical velocity may be 10 cm/s for entraining the unsuspended particles 105 in saline. In this example, when the critical velocity to entrain the unsuspended particles 105 in saline is 10 cm/s the required flow rate of the carrier fluid 104 is 1.2 mL/min for a 0.5 mm inner diameter delivery conduit 410. In another example, the critical velocity may be 3 cm/s for entraining the unsuspended particles 105 in water. In this example, the flow rate of the carrier fluid 104 to entrain the unsuspended particles 105 may be about 4 mL/min for a 1.7 mm inner diameter delivery conduit. In another example a velocity of the carrier fluid 104 may be 6 cm/s. In this example, the flow rate of the carrier fluid 104 to entrain the unsuspended particles 105 may be about 0.66 mL/min for a 0.5 mm inner diameter delivery conduit. It will be appreciated that other flow rates may be chosen depending on the critical velocity necessary to entrain the unsuspended particles 105 and the inner diameter of the delivery conduit 410.

In another aspect, all of the systems 100, 200, 300, 400 described herein may be controlled by a processor. The processor may automate unsuspended particle delivery by automating the actuation of the agitation mechanisms (e.g., rocker plate, cycling syringe system, magnetic mixer assembly, and peristaltic pump) and infusion mechanisms (e.g., infusion syringe plungers) described herein. It will be appreciated that the syringes described herein may be replaced by equivalent systems known in the art. For example, a reservoir and pump system may be used instead of or in combination with the syringes described herein. In some examples, the reservoir may be a vial, a syringe barrel, or any type of container.

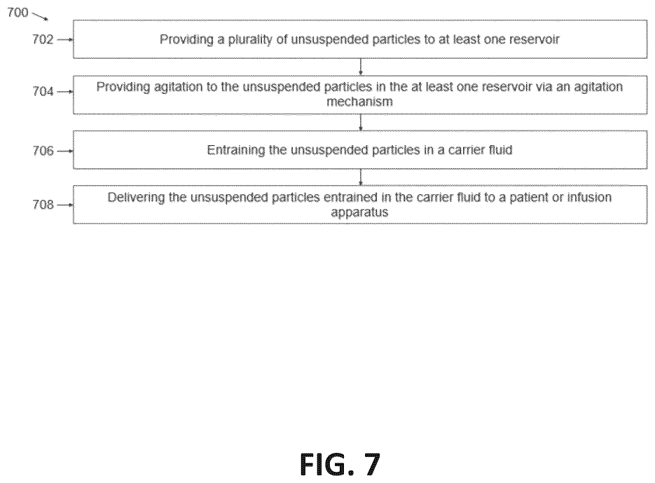

Further described herein is a method for controlled administration of unsuspended particles. The method may use the systems described herein to administer unsuspended particles that are homogenously determined. In other examples, the method may use the systems described herein to administer unsuspended particles at a desired heterogenous dispersion. FIG. 7 illustrates a method 700 for controlled administration of unsuspended particles.

At block 702, the method may include providing a plurality of unsuspended particles to at least one reservoir. The at least one reservoir may be a barrel of syringe, as described herein. In another example, the plurality of unsuspended particles may be placed in another type of reservoir, as described herein (e.g., second reservoir).

At block 704, the method may include providing agitation to the unsuspended particles in the at least one reservoir via an agitation mechanism. In some examples, the agitation mechanism may be a rocker plate, a cycling mechanism (e.g., two syringes), a magnetic mixer assembly, or a peristaltic pump, as described herein. The agitation mechanism may be operable to homogenously or heterogeneously disperse the plurality of unsuspended particles.

At block 706, the method may include entraining the unsuspended particles in a carrier fluid. The unsuspended particles may be entrained in the carrier fluid using the conduits and tubing described herein.

At block 708, the method may include delivering the unsuspended particles entrained in the carrier fluid to a patient or infusion apparatus. The unsuspended particles entrained in the carrier fluid may be delivered to the patient or infusion apparatus via the delivery conduit described herein. In some examples, the unsuspended particles may be delivered homogenously dispersed. In other examples, the unsuspended particles may be delivered at a desired heterogeneous dispersion.

### EXAMPLES

In a first example, unsuspended particles were delivered using the mechanical agitation system 200 of FIGS. 2A-2D. The cycle period was 30 seconds (e.g., the period comprising the time for the first syringe to go from a full volume to zero volume within the barrel). An infusion time of 5 minutes was used with 30 mL of unsuspended particles. FIG. 5 illustrates the delivery rate 500 of the entrained unsuspended particles and the syringe volumes 504, 502 for the first and second syringes.

In a second example, illustrated by FIG. 6, iron oxide nanoparticles (INOP) were delivered to a patient using the mechanical agitation system 200 of FIGS. 2A-2C. The INOP volume was 30 mL. The INOP concentration was 16.7 mg/mL. The cycle period was 30 seconds (e.g., the period comprising the time for the first syringe to go from a volume of 30 mL to zero volume). The total INOP mass delivered was 450 mg. The equivalent INOP volume was 27 mL. The infusion time was 15 minutes. The carrier fluid was saline. The volume of the carrier fluid was 200 mL. The total amount of saline administered was 173 mL. The flow rate of the saline was 11.53 mL/min. A flow rate of INOP for the duration of the infusion was 30 mg/mL. The infusion rate of the unsuspended particles entrained in the saline was 13.3 mL/min. The delivery rate 500 of the entrained unsuspended particles and syringe volumes 504, 502 for the first and second syringes are illustrated in FIG. 6.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

### Exemplary Embodiments

The following is a list of non-limiting exemplary embodiments and may include combinations thereof.

Embodiment 1: A system for controlled administration of unsuspended particles, the system comprising: at least one reservoir operable to contain a plurality of unsuspended particles and/or a carrier fluid; an agitation mechanism operable to agitate the unsuspended particles in the at least one reservoir; and a delivery conduit in fluid communication with the at least one reservoir.

Embodiment 2: The system of embodiment 1, wherein the agitation mechanism provides mechanical agitation to the unsuspended particles to homogenously disperse the plurality of unsuspended particles.

Embodiment 3: The system of embodiment 1, wherein the at least one reservoir is a barrel of a syringe.

Embodiment 4: The system of embodiment 3, wherein syringe further comprises a plunger operable to expel the plurality of unsuspended particles and/or carrier fluid from the barrel into the delivery conduit.

Embodiment 5: The system of embodiment 4, wherein the agitation mechanism is a rocker plate coupled to the barrel.

Embodiment 6: The system of embodiment 5, further comprising one or more agitation bodies within the barrel of the syringe.

Embodiment 7: The system of embodiment 6, wherein the rocker plate agitates the one or more agitation bodies causing mechanical and homogenous dispersion of the unsuspended particles in the barrel.

Embodiment 8: The system of embodiment 7, wherein the agitation mechanism is a cycling mechanism.

Embodiment 9: The system of embodiment 8, wherein the cycling mechanism comprises a first syringe having a barrel and a plunger and a second syringe having a barrel and a plunger, wherein the first syringe is in fluid communication with the second syringe.

Embodiment 10: The system of embodiment 9, further comprising a third syringe having a barrel and a plunger, wherein the barrel of the third syringe is the at least one reservoir containing the carrier fluid.

Embodiment 11: The system of embodiment 10, wherein the barrel of the first syringe and the barrel of the second syringe contain the unsuspended particles.

Embodiment 12: The system of claim 11, further comprising a manifold in fluid communication with the first syringe, the second syringe, the third syringe, and the delivery conduit.

Embodiment 13: The system of embodiment 12, wherein the plunger of the first syringe and the plunger of the second syringe are operable to compress and/or decompress to cycle the unsuspended particles between the first syringe and the second syringe through the manifold causing mechanical and homogenous dispersion of the unsuspended particles.

Embodiment 14: The system of embodiment 13, wherein the plunger of the third syringe is operable to be compressed and expel the carrier fluid into the manifold.

Embodiment 15: The system of embodiment 14, wherein the carrier fluid in the manifold entrains some of the plurality of unsuspended particles, thereby providing the entrained unsuspended particles to the delivery conduit.

Embodiment 16: The system of embodiment 1, wherein the agitation mechanism is a magnetic mixer assembly.

Embodiment 17: The system of embodiment 16, wherein the magnetic mixer assembly comprises a magnetic mixer fan.

Embodiment 18: The system of embodiment 17, wherein the at least one reservoir comprises a syringe having a barrel and a plunger.

Embodiment 19: The system of embodiment 18, wherein the magnetic mixer assembly is coupled to an interior of the barrel of the syringe.

Embodiment 20: The system of embodiment 19, further comprising an external magnet subassembly configured to be rotated causing a change in a magnetic field operable to rotate the magnetic mixer fan.

Embodiment 21: The system of embodiment 20, wherein a rotation of the magnetic mixer fan causes mechanical and homogenous dispersion of the unsuspended particles in the barrel of the syringe.

Embodiment 22: The system of claim 21, wherein the plunger is operable to expel the homogenous dispersed unsuspended particles and the carrier fluid into the delivery conduit.

Embodiment 23: The system of claim 1, wherein the agitation mechanism is a peristaltic pump.

Embodiment 24: The system of embodiment 23, wherein the carrier fluid is in the at least one reservoir and the unsuspended particles are in a peristaltic reservoir.

Embodiment 25: The system of embodiment 24, wherein the peristaltic pump provides a pressure to the unsuspended particles in the peristaltic reservoir causing the unsuspended particles to be homogenously dispersed.

Embodiment 26: The system of embodiment 25, wherein the at least one reservoir is a syringe having a barrel and a plunger in fluid communication with the peristaltic reservoir.

Embodiment 27: The system of embodiment 26, wherein the plunger is operable to provide the carrier fluid to the peristaltic reservoir and entrain the homogenously dispersed unsuspended particles.

Embodiment 28: The system of embodiment 27, wherein the peristaltic reservoir is in fluid communication with the delivery conduit.

Embodiment 29: The system of embodiment 28, wherein the carrier fluid and the entrained unsuspended particles are delivered to the delivery conduit by the pressure provided by the peristaltic pump.

Embodiment 30: The system of embodiment 1, wherein the unsuspended particles are magnetic nanoparticles and the carrier fluid is saline.

Embodiment 31: A system for controlled administration of unsuspended particles, the system comprising: at least one reservoir containing a plurality of unsuspended particles, a carrier fluid, and one or more agitation bodies; an agitation mechanism operable to mechanically agitate the one or more agitation bodies causing the plurality of unsuspended particles to be homogenously dispersed; and a delivery conduit in fluid communication with the at least one reservoir, the delivery conduit operable to deliver the homogenously dispersed unsuspended particles and the carrier fluid to a patient; wherein the agitation mechanism is a rocker plate coupled to the at least one reservoir.

Embodiment 32: A system for controlled administration of unsuspended particles, the system comprising: a first reservoir and a second reservoir containing a plurality of unsuspended particles, the first reservoir in fluid communication with the second reservoir, the first reservoir and the second reservoir in fluid communication with a manifold; a third reservoir containing a carrier fluid, the third reservoir in fluid communication with the manifold; and a delivery conduit in fluid communication with the manifold; wherein the unsuspended particles are cycled between the first reservoir and the second reservoir via a pressure source causing homogenous dispersion of the plurality of unsuspended particles; wherein the carrier fluid is expelled from the third reservoir and entrains some of the plurality of unsuspended particles in the manifold; wherein the entrained unsuspended particles in the carrier fluid are delivered to a patient via the delivery conduit.

Embodiment 33: A system for controlled administration of unsuspended particles, the system comprising: at least one reservoir containing a plurality of unsuspended particles and a carrier fluid; a magnetic mixer fan assembly coupled within the at least one reservoir; an external magnet assembly located near an exterior of the at least one reservoir; and a delivery conduit in fluid communication with the at least one reservoir; wherein rotation of the external magnet assembly causes rotation of the magnet mixer fan; wherein rotation of the magnetic mixer fan assembly causes fluidic mixing of the plurality of unsuspended particles in the at least one reservoir; wherein the fluidically mixed unsuspended particles and the carrier fluid are expelled from the at least one reservoir to the delivery conduit via a pressure source.

Embodiment 34: A system for controlled administration of unsuspended particles, the system comprising: a first reservoir containing a carrier fluid, the first reservoir in fluid communication with a peristaltic reservoir containing a plurality of unsuspended particles; a peristaltic pump in fluid communication with the peristaltic reservoir, the peristaltic pump operable to provide a pressure to the peristaltic reservoir, thereby homogenously dispersing the plurality of unsuspended particles; and a delivery conduit in fluid communication with the peristaltic reservoir; wherein the carrier fluid is provided to the peristaltic reservoir via a pressure source; wherein the carrier fluid entrains the unsuspended particles in the peristaltic reservoir; wherein the carrier fluid and the unsuspended particles are provided to the delivery conduit via the pressure provided by the peristaltic pump.

Embodiment 35: A method for controlled administration of unsuspended particles, the method comprising: providing a plurality of unsuspended particles to at least one reservoir; providing agitation to the plurality of unsuspended particles in the at least one reservoir via an agitation mechanism; entraining the plurality of unsuspended particles in a carrier fluid; and delivering the plurality of unsuspended particles entrained in the carrier fluid to a patient or infusion apparatus via a delivery conduit.

Embodiment 36: A system for controlled administration of unsuspended particulates to a patient comprising: at least one reservoir containing the unsuspended particulates within a carrier fluid at least prior to complete administration of the unsuspended particulates to the patient: an agitator configured to agitate the unsuspended particulates within the carrier fluid to distribute the unsuspended particulates within the at least one reservoir, wherein the agitator comprises one of i) a reciprocating plate coupled to the at least one reservoir configured to reciprocally move the at least one reservoir, ii) a cycling mechanism configured to cycle the unsuspended particulates within the carrier fluid within the at least one reservoir, and iii) a magnetic agitation system configured to change a magnetic field around the at least one reservoir containing the unsuspended particulates within the carrier fluid; a delivery tube fluidly coupled to the at least one reservoir and configured to receive a homogenous mixture of the unsuspended particulates within the carrier fluid for controlled administration of the unsuspended particulates to the patient; and a pressure mechanism fluidly coupled to the at least one reservoir and configured to direct the homogenous mixture of the unsuspended particulates within the carrier fluid to the delivery tube.

Embodiment 37: The system of embodiment 36, wherein the unsuspended particulates are nanoparticles.

Embodiment 38: The system of embodiment 37, wherein the nanoparticles are magnetic nanoparticles.

Embodiment 39: The system of embodiment 36, wherein the agitator comprises the reciprocating plate coupled to the at least one reservoir configured to reciprocally move the at least one reservoir.

Embodiment 40: The system of embodiment 39, wherein only one reservoir containing the unsuspended particulates within the carrier fluid is provided.

Embodiment 41: The system of embodiment 40, wherein the at least one reservoir is formed as a cylindrical body.

Embodiment 42: The system of embodiment 41, wherein the pressure mechanism is a linearly movable plunger received within one end of the cylindrical body.

Embodiment 43: The system of embodiment 42, wherein the delivery tube is coupled to a hub of the cylindrical body.

Embodiment 44: The system of embodiment 43, further including agitation bodies within the cylindrical body that remain within the cylindrical body.

Embodiment 45: The system of embodiment 36, wherein the agitator comprises a cycling mechanism configured to cycle the unsuspended particulates within the carrier fluid within the at least one reservoir.

Embodiment 46: The system of embodiment 45, wherein two reservoirs containing the unsuspended particulates within the carrier fluid are provided and fluidly coupled to each other.

Embodiment 47: The system of embodiment 46, wherein the cycling mechanism includes two linearly movable plungers, each plunger received within one end of one reservoir containing the unsuspended particulates within the carrier fluid.

Embodiment 48: The system of embodiment 47, wherein the pressure mechanism includes a carrier fluid reservoir and a linearly movable plunger received within one end of the carrier fluid reservoir.

Embodiment 49: The system of embodiment 48, wherein the at least one reservoir containing unsuspended particulates within a carrier fluid agitator comprises a fluid loop and the cycling mechanism is configured to cycle the unsuspended particulates within the carrier fluid around the fluid loop.

Embodiment 50: The system of embodiment 49, wherein the fluid loop is formed of tubing and the cycling mechanism is a peristaltic pump.

Embodiment 51: The system of embodiment 50, wherein the pressure mechanism includes a carrier fluid reservoir and a linearly movable plunger received within one end of the carrier fluid reservoir.

Embodiment 52: The system of embodiment 51, wherein the carrier fluid reservoir is coupled to one end of the fluid loop and the delivery tube is coupled to an opposite end of the fluid loop.

Embodiment 53: The system of embodiment 36, wherein the agitator comprises a magnetic agitation system configured to change a magnetic field around the at least one reservoir containing the unsuspended particulates within the carrier fluid.

Embodiment 54: The system of embodiment 53, further including a magnetic rotating element within the at least one reservoir.

Embodiment 55: The system of embodiment 36, wherein the unsuspended particulates are micro-particles.

What is claimed is:

1. A system for controlled administration of unsuspended particles, the system comprising:
at least one reservoir operable to contain a plurality of unsuspended particles and/or a carrier fluid;
an agitation mechanism operable to agitate the unsuspended particles in the at least one reservoir; and
a delivery conduit in fluid communication with the at least one reservoir.

2. The system of claim 1, wherein the agitation mechanism provides mechanical agitation to the unsuspended particles to homogenously disperse the plurality of unsuspended particles.

3. The system of claim 1, wherein the at least one reservoir is a barrel of a syringe.

4. The system of claim 3, wherein the syringe further comprises a plunger operable to expel the plurality of unsuspended particles and/or the carrier fluid from the barrel into the delivery conduit.

5. The system of claim 4, wherein the agitation mechanism is a rocker plate coupled to the barrel.

6. The system of claim 5, further comprising one or more agitation bodies within the barrel of the syringe.

7. The system of claim 6, wherein the rocker plate agitates the one or more agitation bodies causing mechanical and homogenous dispersion of the unsuspended particles in the barrel.

8. The system of claim 7, wherein the agitation mechanism is a cycling mechanism.

9. The system of claim 8, wherein the cycling mechanism comprises a first syringe having a barrel and a plunger and a second syringe having a barrel and a plunger, wherein the first syringe is in fluid communication with the second syringe.

10. The system of claim 9, further comprising a third syringe having a barrel and a plunger, wherein the barrel of the third syringe is the at least one reservoir containing the carrier fluid.

11. The system of claim 10, wherein the barrel of the first syringe and the barrel of the second syringe contain the plurality of unsuspended particles.

12. The system of claim 11, further comprising a manifold in fluid communication with the first syringe, the second syringe, the third syringe, and the delivery conduit.

13. The system of claim 12, wherein the plunger of the first syringe and the plunger of the second syringe are operable to compress and/or decompress to cycle the unsuspended particles between the first syringe and the second syringe through the manifold causing mechanical and homogenous dispersion of the unsuspended particles.

14. The system of claim 13, wherein the plunger of the third syringe is operable to be compressed and expel the carrier fluid into the manifold.

15. The system of claim 14, wherein the carrier fluid in the manifold entrains some of the plurality of unsuspended particles, thereby providing the entrained unsuspended particles to the delivery conduit.

16. The system of claim 1, wherein the agitation mechanism is a magnetic mixer assembly comprising a magnetic mixer fan and an external magnet subassembly configured to be rotated, thereby causing a change in a magnetic field operable to rotate the magnetic mixer fan.

17.-22. (canceled)

23. The system of claim 1, wherein the agitation mechanism is a peristaltic pump.

24.-29. (canceled)

30. The system of claim 1, wherein the unsuspended particles are magnetic nanoparticles and the carrier fluid is saline.

31. A system for controlled administration of unsuspended particles, the system comprising:
at least one reservoir containing a plurality of unsuspended particles, a carrier fluid, and one or more agitation bodies;
an agitation mechanism operable to mechanically agitate the one or more agitation bodies causing the plurality of unsuspended particles to be homogenously dispersed; and
a delivery conduit in fluid communication with the at least one reservoir, the delivery conduit operable to deliver the homogenously dispersed unsuspended particles and the carrier fluid to a patient;
wherein the agitation mechanism is a rocker plate coupled to the at least one reservoir.

32.-34. (canceled)

35. A method for controlled administration of unsuspended particles, the method comprising:
providing a plurality of unsuspended particles to at least one reservoir;
providing agitation to the plurality of unsuspended particles in the at least one reservoir via an agitation mechanism;
entraining the plurality of unsuspended particles in a carrier fluid; and
delivering the plurality of unsuspended particles entrained in the carrier fluid to a patient or infusion apparatus via a delivery conduit.
